SEQUENCE LISTING (1 sequence)

SEQ ID NO: 1 - protein, 338 aa, Unknown
  Antisense target domain
MSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

(19) United States
(12) Patent Application Publication
(10) Pub. No.: US 2023/0218680 A1
(43) Pub. Date: Jul. 13, 2023

(54) METHOD AND COMPOSITION FOR MICROBIOME BASED AMELIORATION OF SKIN ASSOCIATED AUTOIMMUNE INFLAMMATORY DISEASES

(71) Applicant: Tata Consultancy Services Limited, Mumbai (IN)

(72) Inventors: SUNIL NAGPAL, Pune (IN); SUBHRAJIT BHAR, Pune (IN); MOHAMMED MONZOORUL HAQUE, Pune (IN); SHARMILA SHEKHAR MANDE, Pune (IN)

(73) Assignee: Tata Consultancy Services Limited, Mumbai (IN)

(21) Appl. No.: 17/759,734

(22) PCT Filed: Jan. 29, 2021
(86) PCT No.: PCT/IB2021/050717

§ 371 Date: Jul. 29, 2022

(30) Foreign Application Priority Data
Jan. 29, 2020 (IN) 202021004025

(51) Int. Cl. A61K 35/74; A61K 9/70; A61K 35/747; A61K 35/742; A61K 35/741; C12Q 1/689; G16H 10/40

(52) U.S. Cl. CPC A61K 35/74; A61K 9/70; A61K 35/747; A61K 35/742; A61K 35/741; C12Q 1/689; G16H 10/40; C12Q 1/6869

(57) ABSTRACT

Skin-associated autoimmune diseases are common these days. A method and composition for microbiome based amelioration of skin associated autoimmune inflammatory diseases has been provided. The composition is made of at least one or more of microbiome-associated compounds such as proteins, metabolites, antibiotics, probiotics, etc. The method provides a composition for an affected individual through application of these compositions aimed at improving the bioavailability of lipoic acid. It acts through modulation of the lipoic acid metabolic pathway to do the same. The suggested microbes and compounds can either be used as an effective probiotic supplement in increasing the microbial population involved in lipoic acid biosynthesis (only), or increasing the number of competitors of the microbes involved in escalation of lipoic acid salvage system, or through direct antibiotic or physical action against the latter.

### CROSS-REFERENCE TO RELATED APPLICATIONS AND PRIORITY

The present application claims priority from Indian application no. 202021004025, filed on Jan. 29, 2021. The entire contents of the aforementioned application are incorporated herein by reference.

### TECHNICAL FIELD

The embodiments herein generally relate to the field of skin diseases, and, more particularly, to a method and composition for microbiome based amelioration of skin associated autoimmune inflammatory diseases

### BACKGROUND

Skin-associated autoimmune diseases such as to the likes of Atopic Dermatitis and Psoriasis are becoming more common these days as compared to last decade. Atopic dermatitis is a chronic, relapsing skin disease with an increasing rate of prevalence. Psoriasis is a skin disease characterized by hyperplasia of epidermal keratinocytes resulting in thickening of the epidermis and the presence of red raised scaly plaques. Skin-associated autoimmune diseases not only cause physical agony but also profoundly impact the quality of life of the affected individual. The effect on their quality of life in turn causes fatigue, sleep deprivation, activity restriction and depression.

The generally used approaches/methods for improving conditions of skin-associated auto-immune diseases involve application of either steroid-based ointments or anti-inflammatory/hydrating or moisturizing topical agents (containing compounds such as lipoic acid). There are several side effects to improper use of steroid-based medicines such as potential risk of cutaneous atrophy, striae, telangiectasia, and systemic absorption leading to adrenal suppression. Steroid-based medicines can also cause local side effects such as unpleasant sensation when applied in vicinity to eyes or mouth. The efficacy of non-steroidal medicine/agents can also be influenced by personalized nature of skin that can affect the bioavailability of active ingredients of the medicine. Overall, the current limitations involved in the amelioration of skin-associated autoimmune diseases can be potentially overcome by alternative usage of microbes and microbial products which can be provided in the form of probiotics, supplements, antibiotics, prebiotics etc.

### SUMMARY

Embodiments of the present disclosure present technological improvements as solutions to one or more of the above-mentioned technical problems recognized by the inventors in conventional systems. For example, in one embodiment, a method for preparing compositions for amelioration of skin-associated autoimmune inflammatory diseases for a person. Initially, sample is collected from a lesional skin site of the person. Similarly, sample is also collected from a healthy skin site of the person. Further microbial DNA is extracted from the collected healthy skin site and lesional skin site samples, wherein the extracted microbial DNA corresponds to genetic material extracted from a plurality of microbes or a subset of the plurality of microbes inhabiting on the skin of the person. In the next step, microbial sequence data is obtained by performing microbiome sequencing of: the extracted microbial DNA corresponding to the plurality of microbes of the person, or the extracted microbial DNA corresponding to the subset of the plurality of microbes harboring one or more of pathways which are involved in biosynthesis of lipoic acid and upregulation of lipoic acid salvage, wherein the subset of the plurality of microbes mentioned in a first list, a second list, a third list, a fourth list, a fifth list, and a sixth list. Further, microbiome taxonomic profiles are generated from the obtained microbial sequencing data, wherein the microbiome taxonomic profiles indicate one of an absolute abundance and a relative abundance of each of the plurality of microbes inhabiting the lesional skin site or the healthy skin site samples respectively. Further a first ratio of relative abundances of the subset of the plurality of microbes within the microbiome taxonomic profile from the lesional skin site harboring one or more pathways involved in lipoic acid biosynthesis to that of the subset of the plurality of microbes within the microbiome taxonomic profile from the lesional skin site harboring one or more pathways involved in upregulation of lipoic acid salvage is computed. Similarly, a second ratio of relative abundances of the subset of the plurality of microbes within the microbiome taxonomic profile from the healthy skin site harboring one or more pathways involved in lipoic acid biosynthesis to relative abundances of subset of the plurality of microbes within the microbiome taxonomic profile from the lesional skin site harboring the same pathways in lipoic acid biosynthesis is computed. And similarly, a third ratio of relative abundances of subset of the plurality of microbes within the microbiome taxonomic profile from healthy skin site harboring one or more pathways in upregulation of lipoic acid salvage to relative abundances of subset of the plurality of microbes within the microbiome taxonomic profile from lesion skin site harboring the same pathways in upregulation of lipoic acid salvage is computed. In the next step, a first composition is administered to the person if the first ratio is less than one, wherein the first composition comprises one or more of: one or more of microbes enlisted in the first list and the second list as probiotic, one or more compounds inducing favourable physical and chemical factors, anti-sense RNA sequences, or antimicrobials, and wherein the first composition is configured to perform one or more of: promoting the growth of biosynthesis microbes through favourable physical factors and by administration of one or more probiotic non-pathogenic microbes harboring lipoic acid biosynthetic pathway enlisted in the first list and the second list, wherein the biosynthesis microbes refer to one or more microbes harboring one or more lipoic acid biosynthesis pathways, reducing the abundance of salvage microbes through competing microbes and antibiotics that target one or more salvaging microbes involved in upregulation of lipoic acid salvage as enlisted in the third list, wherein the salvage microbes refer to one or more microbes harboring one or more pathways involved in upregulation of lipoic acid salvage, or managing an amplified state through a set of salvage system inhibiting factors enlisted in a seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list and the fifth list and the sixth list are not removed in case they are non-pathogenic commensal microbes. Further, a second composition is administered if the second ratio is more than one, wherein the second composition comprises: one or more of microbes enlisted in the first list and the second list as probiotic, wherein the second composition is configured to perform one or more of: promoting the growth of biosynthesis microbes through favourable physical factors and probiotic non-pathogenic microbes harboring lipoic acid biosynthetic pathway and lacking the salvage pathway as enlisted in the first list and the second list, or promoting the abundance of microbes enlisted in the first list and the second list. And finally, a third composition is administered if the third ratio is less than 1, wherein the third composition comprises: competing microbes against microbes enlisted in the third list, the fourth list and the fifth list, compounds inducing favourable physical and chemical factors, anti-sense RNA sequences, or antimicrobials, wherein the third composition is configured to perform one or more of: managing the amplified state of salvage microbes through competing microbes and antibiotics that target salvaging microbes enlisted in the third list, or managing the amplified state of salvage microbes through salvage system inhibiting factors enlisted in the seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list, the fifth list and the sixth list are not removed in case they are non-pathogenic commensal microbes.

In another aspect, a composition for amelioration of skin-associated autoimmune inflammatory diseases has been provided, the composition comprising one or more of a first composition, a second composition or a third composition. The first composition comprising one or more of: one or more of microbes enlisted in the first list and the second list as probiotic, one or more compounds inducing favourable physical and chemical factors, anti-sense RNA sequences, or antimicrobials, wherein the first composition is administered if a first ratio is less than one. The second composition comprising one or more of microbes enlisted in the first list and the second list as probiotic, wherein the second composition is administered if a second ratio is more than one. The third composition comprising one or more of: competing microbes against microbes enlisted in the third list, the fourth list and the fifth list, compounds inducing favourable physical and chemical factors, anti-sense RNA sequences, or antimicrobials, wherein the third composition is administered if a third ratio is less than one. The first ratio, the second ration, and the third ratio is computed as follows: collecting sample from a lesional skin site of the person; collecting sample from a healthy skin site of the person; extracting microbial DNA from the collected healthy skin site and lesional skin site samples, wherein the extracted microbial DNA corresponds to genetic material extracted from a plurality of microbes or a subset of the plurality of microbes inhabiting on the skin of the person; obtaining microbial sequence data by performing microbiome sequencing of: the extracted microbial DNA corresponding to the plurality of microbes of the person, or the extracted microbial DNA corresponding to the subset of the plurality of microbes harboring one or more of pathways which are involved in biosynthesis of lipoic acid and upregulation of lipoic acid salvage, wherein the subset of the plurality of microbes mentioned in a first list, a second list, a third list, a fourth list, a fifth list, and a sixth list; generating microbiome taxonomic profiles from the obtained microbial sequencing data, wherein the microbiome taxonomic profiles indicate one of an absolute abundance and a relative abundance of each of the plurality of microbes inhabiting the lesional skin site or the healthy skin site samples respectively; computing the first ratio of relative abundances of the subset of the plurality of microbes within the microbiome taxonomic profile from the lesional skin site harboring one or more pathways involved in lipoic acid biosynthesis to that of the subset of the plurality of microbes within the microbiome taxonomic profile from the lesional skin site harboring one or more pathways involved in upregulation of lipoic acid salvage; computing the second ratio of relative abundances of the subset of the plurality of microbes within the microbiome taxonomic profile from the healthy skin site harboring one or more pathways involved in lipoic acid biosynthesis to relative abundances of subset of the plurality of microbes within the microbiome taxonomic profile from the lesional skin site harboring the same pathways in lipoic acid biosynthesis; computing the third ratio of relative abundances of subset of the plurality of microbes within the microbiome taxonomic profile from healthy skin site harboring one or more pathways in upregulation of lipoic acid salvage to relative abundances of subset of the plurality of microbes within the microbiome taxonomic profile from lesion skin site harboring the same pathways in upregulation of lipoic acid salvage.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates a block diagram of a system for preparing compositions for amelioration of skin-associated autoimmune inflammatory diseases according to an embodiment of the present disclosure.

FIGS. 2A-2B is a flowchart illustrating the steps involved in method for preparing compositions for amelioration of skin-associated autoimmune inflammatory diseases according to an embodiment of the present disclosure.

FIG. 3 shows a first implementation of a patch according to an embodiment of the present disclosure.

FIG. 4 shows a second implementation of a patch according to an embodiment of the present disclosure.

FIG. 5 shows a box-plot representation of the abundance of K03800 in antecubital crease (Ac) samples obtained from control, and from atopic dermatitis (AD) affected subjects at various phases of disease severity according to an embodiment of the present disclosure.

FIG. 6 shows a box-plot representation of the abundance of K03800 in popliteal creases (Pc) samples obtained from control, and from Atopic Dermatitis (AD) affected subjects at various phases of disease severity according to an embodiment of the present disclosure.

FIG. 7 shows a box-plot representation of the abundance of K03801 in antecubital crease (Ac) samples obtained from control, and from AD affected subjects in various phases of disease severity according to an embodiment of the present disclosure.

FIG. 8 shows a box-plot representation of the abundance of K03801 in popliteal creases (Pc) samples obtained from control, and from AD affected subjects in various phases of disease severity according to an embodiment of the present disclosure.

FIG. 9 shows a box-plot representation of the abundance of K03644 in antecubital crease (Ac) samples obtained from control, and from AD affected subjects at baseline severity of the disease according to an embodiment of the present disclosure.

FIG. 10 shows a box-plot representation of the abundance of K03800 in Psoriasis samples obtained from control, and from Psoriasis affected subjects from the site of the lesion and any unaffected site according to an embodiment of the disclosure.

FIG. 11 shows a box-plot representation of the abundance of K03801 in Psoriasis samples obtained from control, and from Psoriasis affected subjects from the site of the lesion and any unaffected site according to an embodiment of the disclosure.

### DETAILED DESCRIPTION OF EMBODIMENTS

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Lipoic acid based topical ointments are relatively less harmful than steroid-based medicines. Yet, application of a lipoic acid based topical ointment has its own limitations as its efficacy in skin-penetration as well as its bioavailability after application on skin can be affected by various factors (such as other compounds in the ointment, the base of the ointment being water/oil etc., the environmental factors such as photo-stability, drying of skin, perspiration etc.). Although, existing state of art attempts to address the said limitations by focusing on methods that improve the stability of Lipoic acid against the aforementioned factors. Not much attention has been paid to biotic factors associated with the host skin that can enhance or degrade the efficacy of Lipoic acid. One such biotic factor is ‘biodegradation of alpha Lipoic acid by microbes on the skin through the Lipoic acid metabolic pathway using the Lipoic acid salvage system.

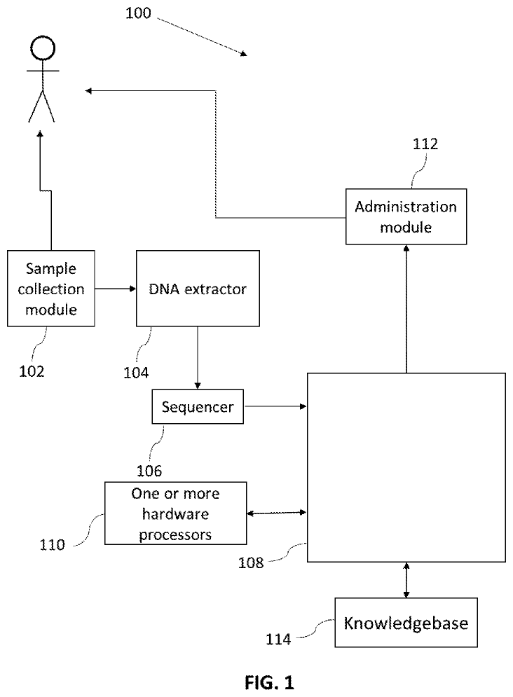

Referring now to the drawings, and more particularly to FIG. 1 through FIG. 11, where similar reference characters denote corresponding features consistently throughout the figures, there are shown preferred embodiments and these embodiments are described in the context of the following exemplary system and/or method.

According to an embodiment of the disclosure, a block diagram of a system 100 for preparing the composition for amelioration skin-associated autoimmune inflammatory diseases is shown in FIG. 1. The present disclosure provides the method and composition for microbiome based amelioration of skin associated autoimmune inflammatory diseases. The composition is made of at least one or more of microbiome-associated compounds such as proteins, metabolites, antibiotics, probiotics, etc. in skin-associated autoimmune diseases such as to the likes of Atopic dermatitis and Psoriasis. The method provides a composition for an affected individual through application of these compositions aimed at improving the bioavailability of lipoic acid. It acts through modulation of the lipoic acid metabolic pathway to do the same.

According to an embodiment of the disclosure, the disclosure provides a solution that involves (a) characterizing the capability of skin associated microbes in lipoic acid metabolism b) validating differential capacity of inflammatory skin-associated microbes in lipoic acid salvage as compared to healthy skin microbes, (c) and further suggest specific microbes and microbe-associated compounds in modulation of the lipoic acid metabolic pathway. The suggested microbes and compounds can either be used as an effective probiotic supplement in increasing the microbial population involved in lipoic acid biosynthesis (only), or increasing the number of competitors of the microbes involved in escalation of lipoic acid salvage system, or through direct antibiotic or physical action against the latter. The solution also suggests use of ligand adjunct based allosteric inhibition of lipoic acid salvage system enzymes and/or the use of microbes that can produce such ligands.

The present disclosure states that lipoic acid/lipoic acid based compound can be effectively utilized as an agent which can improve condition of skin in case of skin-associated autoimmune inflammatory diseases through anti-inflammatory effect and hydration. The disclosure presents an understanding of the lipoic acid metabolic pathway associated enzymes and lipoic acid salvage system. Functional analysis of the system (and its components) indicates a significant difference between individuals who have a healthy skin microbiome as compared to individuals affected with skin-associated autoimmune inflammatory diseases. According to another embodiment of the disclosure, the microbial therapeutics can also be used as a cosmetic solution to address lipoic acid bioavailability and to improve on its application as an anti-ageing compound against skin-ageing associated manifestations.

The method involves identifying, mining and deciphering the protein domains and unique combinations of such domains that are involved with the lipoic acid metabolic pathway in specific skin associated microbes. Based on recognized protein domains and associated microbes, the method suggests specific microbes/microbial concoction and microbial products which can increase the population of lipoic acid synthesizing microbes, or increase the number of microbial competitors of the microbes involved in up-regulation of lipoic acid salvage system as an effective probiotic supplement, direct antibiotic based inhibition of the latter; anti-sense RNA based/ligand adjunct based allosteric inhibition of lipoic acid salvage system enzymes and/or the use of microbes that can produce such ligands, thus improving the bioavailability of the lipoic acid/lipoic acid based compound.

According to an embodiment of the disclosure, the system 100 consists of a sample collection module 102, a DNA extractor 104, a sequencer 106, a memory 108, one or more hardware processors (referred as a processor, herein after) 110 and an administration module 112 as shown in FIG. 1. The processor 110 is in communication with the memory 108. The memory 108 further includes a plurality of modules for performing various functions.

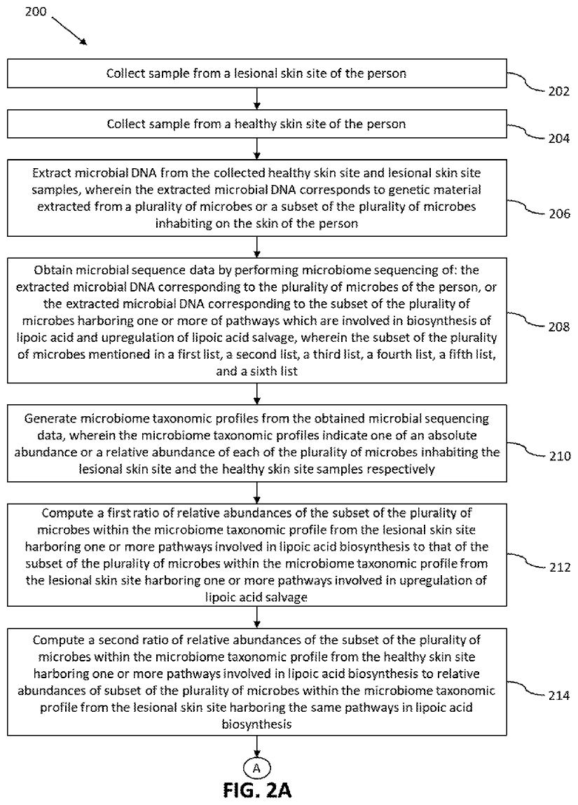

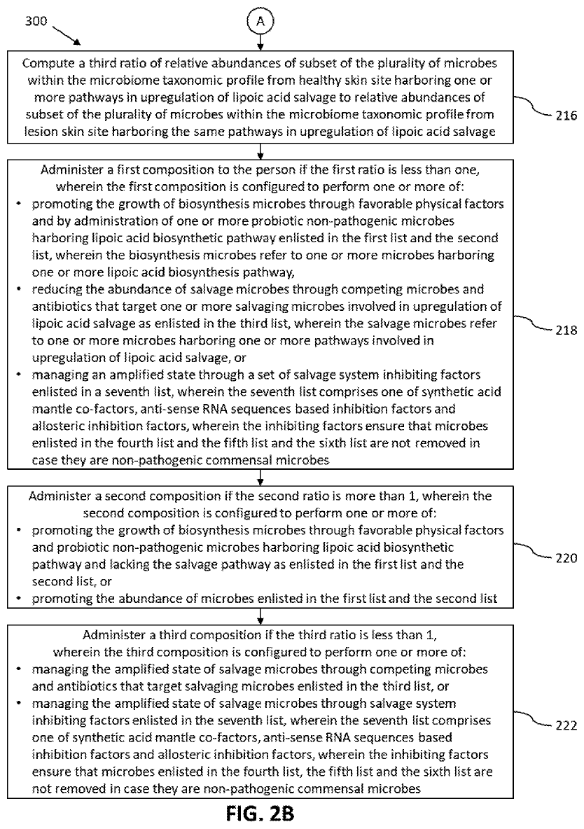

In operation, a flowchart 200 illustrating the steps involved for preparing compositions for amelioration of skin-associated autoimmune inflammatory diseases for a person is shown in FIG. 2A-2B. Initially at step 202, sample is collected from a lesional skin site of the person using the sample collection module 102. Similarly, at step 204, the sample is collected from a healthy skin site of the person using the sample collection module 102. It should be appreciated that the use of any known technique for the sample collection is well within the scope of this disclosure.

Further at step 206, microbial DNA is extracted from the collected healthy skin site and lesional skin site samples using the DNA extractor 104. The extracted microbial DNA corresponds to genetic material extracted from a plurality of microbes or a subset of the plurality of microbes inhabiting on the skin of the person. At step 208, microbial sequence data is obtained by performing microbiome sequencing of: the extracted microbial DNA corresponding to the plurality of microbes of the person. Microbiome sequencing is also performed of the extracted microbial DNA corresponding to the subset of the plurality of microbes harboring one or more of pathways which are involved in biosynthesis of lipoic acid and upregulation of lipoic acid salvage, wherein the subset of the plurality of microbes mentioned in a first list, a second list, a third list, a fourth list, a fifth list, and a sixth list. The first list, the second list, the third list, the fourth list, the fifth list, and the sixth list contains a set of microbes performing different functions as explained in later part of the disclosure. The microbiome sequencing is done either by 16S amplicon sequencing, WGS or targeted qPCR based sequencing of specific microbes.

The sequencing of the microbial DNA is performed using the sequencer 106. The isolated microbial DNA, after purification is subjected to NGS (Next Generation Sequencing) technology for generating human readable form of short stretches of DNA sequence called reads. The said NGS technology involves amplicon sequencing targeting bacterial marker genes (such as 16S rRNA, 23S rRNA, rpoB, cpn60 etc.). The sequence reads, thus obtained, are computationally analysed through widely accepted standard frameworks for NGS data analysis. In another embodiment, the sequencer 106 may involve Whole Genome Sequencing (WGS) where the reads are generated for the total DNA content of a given sample. In yet another embodiment, the set of microbial genes involved in the production of the neuroactive compounds (under the current invention) may be sequenced using targeted PCR (Polymerase Chain Reaction). In yet another implementation, RNA-seq. technology may be used to sequence the microbial RNA (Ribonucleic acid) content of a given sample. This can be performed targeting the whole bacterial RNA content or a particular set of RNAs. RNA-seq provides insights into the active microbial genes in a sample.

Further at step 210, microbiome taxonomic profiles are generated from the obtained microbial sequencing data. The microbiome taxonomic profiles indicate one of an absolute abundance or a relative abundance of each of the plurality of microbes inhabiting the lesional skin site and the healthy skin site samples respectively.

According to an embodiment of the disclosure, the samples can be utilized in couple of scenarios. In the first scenario, only lesional site sample is utilized. While in the second scenario, both lesional and healthy skin site sample can be used. At step 212, a first ratio of relative abundances of the subset of the plurality of microbes within the microbiome taxonomic profile from the lesional skin site harboring one or more pathways involved in lipoic acid biosynthesis to that of the subset of the plurality of microbes within the microbiome taxonomic profile from the lesional skin site harboring one or more pathways involved in upregulation of lipoic acid salvage is computed.

At step 214, a second ratio of relative abundances of the subset of the plurality of microbes within the microbiome taxonomic profile from the healthy skin site harboring one or more pathways involved in lipoic acid biosynthesis to relative abundances of subset of the plurality of microbes within the microbiome taxonomic profile from the lesional skin site harboring the same pathways in lipoic acid biosynthesis is computed.

Similarly at step 216, a third ratio of relative abundances of subset of the plurality of microbes within the microbiome taxonomic profile from healthy skin site harboring one or more pathways in upregulation of lipoic acid salvage to relative abundances of subset of the plurality of microbes within the microbiome taxonomic profile from lesion skin site harboring the same pathways in upregulation of lipoic acid salvage is computed. The first ratio, the second ratio and the third ratio is further utilized to determine which composition needs to be administered to the person.

At step 218, a first composition is administered to the person if the first ratio is less than one using the administration module 112. The first ratio of less than one indicates either a depletion of biosynthetic pathway microbes or over-abundance of salvage microbes. The first composition comprises one or more of: one or more of microbes enlisted in the first list and the second list as probiotic, one or more compounds inducing favourable physical and chemical factors, anti-sense RNA sequences, or antimicrobials.

According to an embodiment of the disclosure, the first composition is configured to perform one or more of:
promoting the growth of biosynthesis microbes through favourable physical factors and by administration of one or more probiotic non-pathogenic microbes harboring lipoic acid biosynthetic pathway enlisted in the first list and the second list, wherein the biosynthesis microbes refer to one or more microbes harboring one or more lipoic acid biosynthesis pathway,
reducing the abundance of salvage microbes through competing microbes and antibiotics that target one or more salvaging microbes involved in upregulation of lipoic acid salvage as enlisted in the third list, wherein the salvage microbes refer to one or more microbes harboring one or more pathways involved in upregulation of lipoic acid salvage, or
managing an amplified state through a set of salvage system inhibiting factors enlisted in a seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list and the fifth list and the sixth list are not removed in case they are non-pathogenic commensal microbes;

At step 220, a second composition is administered if the second ratio is more than 1 using the administration module 112. The second composition comprises: one or more of microbes enlisted in the first list and the second list as probiotic. The second composition is configured to perform one or more of:
promoting the growth of biosynthesis microbes through favourable physical factors and probiotic non-pathogenic microbes harboring lipoic acid biosynthetic pathway and lacking the salvage pathway as enlisted in the first list and the second list, or
promoting the abundance of microbes enlisted in the first list and the second list; and

Finally at step 222, a third composition is administered if the third ratio is less than 1 using the administration module 112. The third composition comprises: competing microbes against microbes enlisted in the third list, the fourth list and the fifth list, compounds inducing favourable physical and chemical factors, anti-sense RNA sequences, or antimicrobials, wherein the third composition is configured to perform one or more of:
managing the amplified state of salvage microbes through competing microbes and antibiotics that target salvaging microbes enlisted in the third list, or
managing the amplified state of salvage microbes through salvage system inhibiting factors enlisted in the seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list, the fifth list and the sixth list are not removed in case they are non-pathogenic commensal microbes.

According to an embodiment of the disclosure, the first list is a set of non-pathogenic microbes capable of thriving on skin and synthesizing lipoic acid, the second list is a set of non-pathogenic lipoic acid synthesizing microbes other than that mentioned in first list, the third list is a set of pathogenic salvage microbes which can be targeted by antibiotics, the fourth list is a set of skin inhabiting microbes capable of degrading lipoic acid, the fifth list is a set of skin inhabiting microbes, capable of degrading lipoic acid, other than those listed in the fourth list, the sixth list is a set of non-pathogenic skin inhabiting microbes capable of synthesizing as well as degrading lipoic acid, and the seventh list is one or more of synthetic acid mantle co-factors, anti-sense RNA target sequence based inhibition factors and allosteric inhibition factors.

According to an embodiment of the disclosure, the first list comprises one or more of: Corynebacterium efficiens, Corynebacterium glutamicum, Corynebacterium variabile, Corynebacterium callunae, Propionibacterium freudenreichii, Burkholderia vietnamiensis, Pseudomonas poae, Methylobacillus flagellatus, Erwinia tasmaniensis, Erwinia billingiae, Rhodoferax ferrireducens, Cupriavidus necator.

According to an embodiment of the disclosure, the second list comprises one or more of: Brachybacterium faecium, Corynebacterium efficiens, Corynebacterium glutamicum, Corynebacterium halotolerans, Salinispora tropica, Streptosporangium roseurn, Corynebacterium variabile, Mycobacterium gilvum, Rubrobacter xylanophilus, Cellulomonas flavigena, Corynebacterium callunae, Sulfobacillus acidophilus, Microbacterium testaceum, Mycobacterium vanbaalenii, Mycobacterium indicuspranii, Propionibacterium freudenreichii, Nitrosococcus oceani, Nitrosococcus halophilus, Methylibium petroleiphilum, Methyloteneram obilis, Marinomonas mediterranea, Marinomonas posidonica, Cupriavidus necator, Shewanella amazonensis, Rhodoferax ferrireducens, Shewanella oneidensis, Shewanella woodyi, Shewanella violacea, Burkholderia vietnamiensis, Erwinia billingiae, Erwinia tasmaniensis, Thioalkalimicrobium cyclicum, Ramlibacter tataouinensis, Polaromonas naphthalenivorans, Alicycliphilus denitrificans, Aliivibrio fischeri, Buchnera aphidicola, Chromohalobacter salexigens, Psychromonas ingrahamii, Tolumonas auensis, Ferrimonas balearica, Halomonas elongate, Idiomarina loihiensis, Halothiobacillus neapolitanus, Methylobacillus flagellates, Thiomicrospira crunogena, Pseudomonas poae, Photobacterium profundum and Leptothrix cholodnii.

According to an embodiment of the disclosure, the third list comprises one or more of: Acholeplasma brassicae, Acholeplasma oculi, Acholeplasma palmae, Achromobacter denitrificans, Achromobacter xylosoxidans, Aerococcus urinae, Aerococcus urinaeequi, Aeromonas hydrophila, Aeromonas salmonicida, Aeromonas veronii, Bacillus anthracis, Bacillus cereus, Bacillus cytotoxicus, Bacillus infantis, Bacillus subtilis, Bacillus thuringiensis, Bordetella avium, Bordetella petrii, Brevibacillus brevis, Clostridioides difficile, Clostridium botulinum, Clostridium saccharolyticum, Clostridium tetani, Cronobacter condimenti, Cronobacter malonaticus, Cronobacter turicensis, Enterococcus casseliflavus, Enterococcus faecalis, Enterococcus faecium, Enterococcus hirae, Erysipelothrix larvae, Erysipelothrix rhusiopathiae Pathogenic, Escherichia fergusonii, Halobacteriovorax marinus, Klebsiella pneumoniae, Kluyvera intermedia, Lactobacillus fermentum, Lactobacillus parabuchneri, Leclercia adecarboxylata, Leptotrichia buccalis, Leuconostoc garlicum, Leuconostoc lactis, Leuconostoc mesenteroides, Listeria ivanovii, Listeria monocytogenes, Macrococcus caseolyticus, Melissococcus plutonius, Mesoplasma forum, Mycoplasma agalactiae, Mycoplasma bovis, Mycoplasma bovoculi, Mycoplasma capricolum, Mycoplasma cynos, Mycoplasma fermentans, Mycoplasma gallisepticum, Mycoplasma genitalium, Mycoplasma hyopneumoniae, Mycoplasma leachii, Mycoplasma mobile, Mycoplasma mycoides, Mycoplasma penetrans, Mycoplasma pneumoniae, Mycoplasma putrefaciens, Mycoplasma synoviae, Paenibacillus larvae, Pantoea agglomerans, Pantoea ananatis, Pectobacterium carotovorum, Prevotella enoeca, Prevotella intermedia, Rahnella aquatilis, Raoultella ornithinolytica, Salmonella enterica, Shigella boydii, Shigella dysenteriae, Shigella flexneri, Shigella sonnei, Spiroplasma apis, Spiroplasma citri, Spiroplasma culicicola, Spiroplasma mirum, Staphylococcus aureus, Staphylococcus haemolyticus, Staphylococcus lugdunensis, Staphylococcus pasteuri, Staphylococcus pseudintermedius, Staphylococcus saprophyticus, Streptobacillus moniliformis, Streptococcus acidominimus, Streptococcus anginosus, Streptococcus constellatus, Streptococcus dysgalactiae, Streptococcus equi, Streptococcus gallolyticus, Streptococcus gordonii, Streptococcus infantarius, Streptococcus iniae, Streptococcus lutetiensis, Streptococcus mitis, Streptococcus mutans, Streptococcus oralis, Streptococcus parasanguinis, Streptococcus parauberis, Streptococcus pasteurianus, Streptococcus pneumoniae, Streptococcus pyogenes, Streptococcus sanguinis, Streptococcus suis, Streptococcus uberis, Thermoanaerobacterium thermosaccharolyticum, Treponema denticola, Treponema pedis, Yersinia enterocolitica, Yersinia pestis, Yersinia pseudotuberculosis.

According to an embodiment of the disclosure, the fourth list comprises one or more of: Auricoccus indicus, Staphylococcus epidermidis, Pediococcus acidilactici, Clostridium kluyveri, Lactobacillus brevis, Lactobacillus buchneri, Lactobacillus koreensis, Lactococcus lactis, Streptococcus agalactiae.

According to an embodiment of the disclosure, the fifth list comprises one or more of: Acetohalobium arabaticum, Acholeplasma laidlawii, Aerococcus urinaehominis, Auricoccus indicus, Bacillus amyloliquefaciens, Bacillus atrophaeus, Bacillus cellulosilyticus, Bacillus clausii, Bacillus coagulans, Bacillus halodurans, Bacillus licheniformis, Bacillus megaterium, Bacillus mycoides, Bacillus pumilus, Bacillus toyonensis, Bacillus velezensis, Bdellovibrio bacteriovorus, Carnobacterium maltaromaticum, Carnobacterium sp., Clostridium propionicum, Deinococcus proteolyticus, Desulfarculus baarsii, Desulfitobacterium hafniense, Desulfitobacterium metallireducens, Desulfomicrobium baculatum, Desulfotomaculum ruminis, Desulfovibrio magneticus, Enterococcus mundtii, Escherichia coli, Eubacterium limosum, Exiguobacterium antarcticum, Exiguobacterium sibiricum, Glutamicibacter arilaitensis, Halothermothrix orenii, Hydrogenobacter thermophiles, Ilyobacter polytropus, Intrasporangium calvum, Kosakonia oryzae, Lactobacillus acidipiscis, Lactobacillus casei, Lactobacillus paracasei, Lactobacillus paraplantarum, Lactobacillus plantarum, Lactobacillus reuteri, Lactobacillus rhamnosus, Lactobacillus sakei, Lactobacillus salivarius, Leadbetterella byssophila, Leuconostoc carnosum, Leuconostoc citreum, Leuconostoc gelidum, Leuconostoc kimchi, Listeria innocua, Listeria seeligeri, Listeria welshimeri, Lysinibacillus sphaericus, Meiothermus silvanus, Oenococcus oeni. Oscillibacter valericigenes, Owenweeksia hongkongensis, Paenibacillus bovis, Paenibacillus mucilaginosus, Paenibacillu sterrae, Pantoe avagans, Pediococcus pentosaceus, Planococcus donghaensis, Shimwellia blattae, Sporosarcina psychrophila, Staphylococcus carnosus, Staphylococcus epidermidis, Staphylococcus piscifermentans, Staphylococcus warneri, Streptococcus salivarius, Streptococcus thermophiles, Symbiobacterium thermophilum, Syntrophomonas wolfei, Tetragenococcushalophilus, Weissellakoreensis, Yersinia intermedia, Zunongwangia profunda.

According to an embodiment of the disclosure, the sixth list comprises one or more of: Clostridium kluyveri, Clostridium ljungdahlii, Oceanobacillus iheyensis, Actinobacillus succinogenes and Enterobacter lignolyticus

According to an embodiment of the disclosure, the seventh list comprises one of synthetic acid mantle co-factors, anti-sense RNA sequences based inhibition factors and allosteric inhibition factors, wherein the inhibiting factors ensure that microbes enlisted in the fourth list and the fifth list and the sixth list are not removed in case they are non-pathogenic commensal microbes. Further, acid mantle co-factors composed specifically of Water, Lactic acid, Urocanic acid, C6H & C18:2 fatty acids, Pyrrolidine carboxylic acid, Asparatic acid, Glutamic acid with a pH attainment between 4-4.2. The enzyme (6.3.1.20) involved in salvage system cannot function below pH of 4.2. C6H and C18:2 are selected because of being anti-pathogenic fatty acids and hence useful for skin.

Further, following Mg²⁺ chelator can be used: o-aminophenol-N,N-diacetate-O-methylene-methylphosphinate referred to as APDAP. Moreover, following Mg²⁺ transporter blockers can be used: Zn²⁺, ca²⁺, Mn²⁺, Sr²⁺, Co²⁺

The anti-sense RNA targeting technology is configured to transform an antisense nucleotide sequence as a construct into the target bacteria, wherein the target bacteria refers to bacteria whose lipoic acid salvage pathway is required to be inhibited without killing the said target bacteria, and wherein the antisense nucleotide sequence targets a protein domain with the following amino acid sequence:

MSTLRLLISDSYDPWFNLAVEECIFRQMPATQRVLFLWRNADTVVIGRA
QNPWKECNTRRMEEDNVRLARRSSGGGAVFHDLGNTCFTFMAGKPEYDK
TISTSIVLNALNALGVSAEASGRNDLVVKTVEGDRKVSGSAYRETKDRG
FHHGTLLLNADLSRLANYLNPDKKKLAAKGITSVRSRVTNLTELLPGIT
HE25QVCEAITEAFFAHYGERVEAEIISPNKTPDLPNFAETFARQSSWE
WNFGQAPAFSHLLDERFTWGGVELHFDVEKGHITRAQVFTDSLNPAPLE
ALAGRLQGCLYRADMLQQECEALLVDFPEQEKELRELSAWMAGAVR,

wherein mentioned letters in the amino acid sequence represent various amino acids that constitute the sequence of a protein domain.

According to an embodiment of the disclosure, the microbes enlisted in the first list, the second list, the third list, the fourth list, the fifth list, and the sixth list are identified through following steps. Initially, the plurality of skin inhabiting/capable of inhabiting microbes is determined using one or more of laboratory based (in-vivo/in-vitro) or mining or machine learning techniques or other in-silico methods. Further, genomes of the enlisted plurality of skin inhabiting microbes are profiled to identify a plurality of proteins involved in one or more of lipoic acid biosynthesis pathways or lipoic acid salvage pathways in the genomes. In the next step a plurality of protein domains is identified corresponding to each of the identified plurality of proteins. A domain matrix is then created, wherein column headers of the domain matrix correspond to individual enlisted genomes and row indices correspond to identified protein domains, and wherein a value of one is assigned to the enlisted genome if a domain out of the plurality of domains is present in the said genome else a value of zero is assigned.

Further, a pathway matrix is derived from the domain matrix, wherein for each of the given genomes represented in the domain matrix, a specific pathway is ascertained to be expressed in the genome if all domain(s) constituting the pathway indicate a value of 1 in the created domain matrix. In the next step, using the derived domain matrix and the pathway matrix, the microbes are identified which are involved in different branch points of lipoic acid metabolism using a decision-making system derived knowledgebase 114, wherein the decision-making system derived knowledgebase 114 contains different branch point definitions for different skin microbes, and wherein the mentioned branch point definitions refer to a

(1) capability of a microbe to only synthesize lipoic acid
(2) capability of a microbe to salvage lipoic acid
(3) capability of a microbe to both synthesize and salvage lipoic acid, wherein the branch point definitions are derived using a pattern of co-occurrence of the domains constituting the lipoic acid pathway as well as their presence in genomic neighborhood of each other, and wherein genomic neighborhood signifies the importance of co-occurrence of two domains in proximity to each other for the pathway to be functional.

The compounds in the seventh list comprises one of synthetic acid mantle co-factors, anti-sense RNA sequences based inhibition factors and allosteric inhibition factors.

According to an embodiment of the disclosure, the method for identifying the microbes and compounds enlisted in the first list, the second list, the third list, the fourth list, the fifth list and the sixth list can further be explained in detail as follows. The method is configured to identify bacterial strains capable of colonizing on skin surface and possessing either lipoic acid biosynthesis or degradation capabilities or those having both these metabolic functions. The proteins involved in lipoic acid biosynthesis as well as salvage pathways were identified. The organisms involved, in which these pathways were experimentally characterized, were also noted. Protein fasta sequences corresponding to these proteins were obtained using the databases present in National Centre for Biotechnology Information. The use of any other database for obtaining these sequences is well within the scope of the invention.

The lipoic acid biosynthesis pathway involved proteins LipA and LipB and the corresponding protein fasta files were obtained as mentioned above. These fasta sequences could be used for homology-based searches for presence of these proteins and identifying lipoic acid biosynthesis capability within other microbes. Further Hidden Markov Model (HMM) based search of these proteins was utilized to identify protein domain HMMs representative of these proteins. In this embodiment, Protein Family database or PFAM database was used to identify protein domains present in these proteins. The domains were identified to be LIAS_N for LipA and BPL_lplA_lipB for LipB. The occurrence of these two proteins was found to be in genomic neighbourhood of each other within the organisms the pathway was experimentally characterized in. These domains have been utilized to search presence of lipoic acid biosynthesis genetic machinery in other microbial genomes. The use of any other domain or homology based annotation methods are well within the scope of this invention.

Similarly, the salvage pathways were found to constitute lipoprotein ligase protein lplA whose protein fasta sequence was also obtained as discussed above. Annotation based on HMM based PFAM method indicated presence of BPL_lplA_LipB and Lip_prot_lig_C.

The protein region comprising the PFAM domain BPL_lplA_LipB (INDOM) is the catalytic domain of the enzyme. It should be noted that LplA and LipB contain same domain BPL_lplA_LipB (INDOM) and therefore they should be distinguished correctly during annotation. In one embodiment, a check on the presence of LipA in vicinity of LipB on the microbial genome is necessary to identify biosynthesis pathway accurately. In another embodiment, the salvage enzyme LplA contains an extra protein domain (EXDOM) annotated in PFAM as Lip_prot_lig_C. In yet another embodiment, the EXDOM domain was identified using homology based search where protein fasta sequence obtained earlier for LplA will only show a complete match with LplA sequences while a partial match will be obtained with LipB where the EXDOM is missing. In another embodiment, presence of Glycine Cleavage Domain (GCV) in flanks of LplA further confirms presence of salvage system. Therefore, these domains termed as EXDOM and GCV have been utilized to distinguish the biosynthesis enzyme LipB from salvage enzyme LplA. These extra domains can be identified using any other functional annotation methods. These methods may include but are not limited to homology based annotations, HMM based annotations etc. In all cases, presence of this extra domain can be used as an indicator of salvage LplA enzyme. The same is explained in TABLE 1.

TABLE 1
Functional annotation of various microbes against various domains

| | | | Only | Only | | Only | |
|---|---|---|---|---|---|---|---|
| Pathway decision > | | None | Biosynthesis | salvage | None | salvage | [symbol] |
| Microbes > | | Microbe1 | Microbe2 | Microbe3 | Microbe4 | Microbe5 | [symbol] |
| Domains | LIAS_N | YES | YES | NO | YES | NO | [symbol] |
| | BPL_lplA_lipB | YES | YES | YES | NO | YES | [symbol] |
| | Lip_prot_lig_C | NO | NO | YES | NO | YES | [symbol] |
| | GCV | NO | NO | NO | NO | YES | [symbol] |
| Decision reason | | Domains | Domains of | Since | Comple- | LipA absent | [symbol] |
| | | not in | LipA and | Lip_prot_ | menting | as LIAS_N | |
| | | neighbor- | LipB are in | ligC fron | and | not | |
| | | hood | neighbor- | lplA is | catalytic | discovered | |
| | | (neighbor- | hood and | present in | domains | and even if | |
| | | hood | EXDOM | addition | absent | INDOM is | |
| | | information | absent | to the | | from LipB, | |
| | | is recorded | | INDOM | | biosynthesis | |
| | | separately) | | | | not possible | |
| | | | | | | without | |
| | | | | | | LipA | |

[symbol]

indicates data missing or illegible when filed

Protein fasta sequences of the sequenced bacterial genomes were obtained using the database available in National Centre for Biotechnology Information. The use of any other genome sequence database is also well within scope of this invention

Functional annotation of the microbial genome sequences to identify presence LipA, LipB and LplA proteins and thereby determine Lipoic acid salvage/biosynthesis potential of these microbial organisms. This genome annotation can be performed using homology based methods (BLAST etc.), Hidden Markov Model based methods (PFAM, SMART database etc.), position specific scoring matrices (Conserved domain database etc.) etc. Any other method of protein function annotation on microbial genomes is well within the scope of the invention.

Functional annotation of LipA and LipB proteins further involved checking that these two proteins were located in genomic neighbourhood (within 2-3 genes of each other) of each other within the microbial genomes analyzed.

An organism protein domain matrix was created utilizing organism/genome list as rows and protein domains as columns. Another pathway matrix was created with the organism/genome list as rows and biosynthesis pathway and salvage pathway as columns. It must be noted that the pathway matrix is inferred from the protein domain matrix. Value of 1 is assigned if a domain/pathway is present in an organism's genome, else a value of 0 is assigned.

For all organisms which form the rows in organism pathway matrix, their capability of colonizing on the skin was determined. This information was added as a new column to the Organism Pathway matrix. The organism pathway matrix so created can be utilized as a knowledgebase 114.

The organisms with value of lipoic acid biosynthesis pathways as 1 in organism pathway matrix that have capability of colonizing on skin can be utilized to create microbial consortia/cocktail for production of lipoic acid on skin.

According to an embodiment of the disclosure, the anti-sense RNA targeting technology is configured to transform an antisense nucleotide sequence as a construct into the target bacteria, wherein the target bacteria refers to bacteria whose lipoic acid salvage pathway is required to be inhibited without killing the said target bacteria, and wherein the antisense nucleotide sequence targets a protein domain with the following amino acid sequence:

MSTLRLLISDSYDPWFNLAVEECIFRQMPATQRVLFLWRNADTVVIGRA
QNPWKECNTRRMEEDNVRLARRSSGGGAVFHDLGNTCFTFMAGKPEYDK
TISTSIVLNALNALGVSAEASGRNDLVVKTVEGDRKVSGSAYRETKDRG
FHHGTLLLNADLSRLANYLNPDKKKLAAKGITSVRSRVTNLTELLPGIT
HE25QVCEAITEAFFAHYGERVEAEIISPNKTPDLPNFAETFARQSSWE
WNFGQAPAFSHLLDERFTWGGVELHFDVEKGHITRAQVFTDSLNPAPLE
ALAGRLQGCLYRADMLQQECEALLVDFPEQEKELRELSAWMAGAVR,

wherein mentioned letters in the amino acid sequence represent various amino acids that constitute the sequence of a protein domain.

In one embodiment, this antisense nucleotide sequence can be transformed as a construct into the target bacteria. The construct can further be constituted to contain nucleotide cleaving enzyme which can recognize the EXDOM bound antisense sequence and create a double strand break. This can be followed by an enzyme which can recognize a double strand break and extend the cleavage to flanking catalytic domain (INDOM) of LplA enzyme. Any other method of targeting this nucleotide sequence or protein domain encoded by it is well within scope of this disclosure. The targeting of EXDOM can allow to selectively debilitating organisms capability of salvaging lipoic acid without any cross reactivity with the biosynthesis enzyme which lacks the EXDOM.

The above approach was followed by first identifying more than 900 genomes that were capable of inhabiting the human skin. The entire genome mining was performed on this set of genomes to identify biosynthetic and degrading species. Pathogenicity of the identified genomes was also determined to identify and filter relevant set of microbes.

According to an embodiment of the disclosure, the therapeutic solutions can be applied in at least one of the following forms:
As topical ointment to be applied on skin, in form of paste/liquid/gel
As a spray, roller or other form of application involving delivery of a layer on top of the affected site
As a patch which can be worn temporarily (explained in detail in the later part of this disclosure) as an injection applied in subcutaneous layer or elsewhere
In powder form to be applied as a layer on affected and unaffected skin
Probiotic and prebiotic supplements (microbes/microbial products/chemicals) which can be in form of drinks and which boosts the population of the intended microbes
Any other mode of delivery other than above mentioned which would be applicable to affected and/or unaffected areas of skin

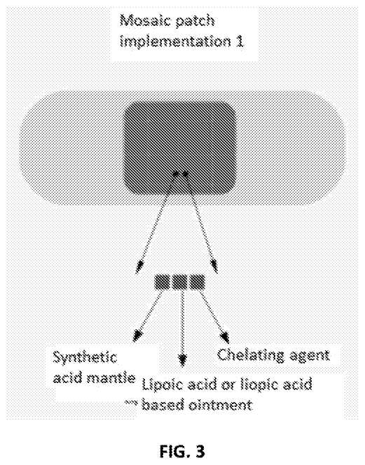

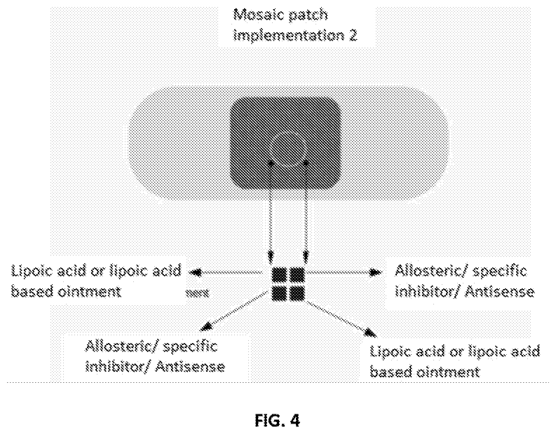

According to an embodiment of the disclosure, the concoction or composition can be applied in the form of patches in first and a second implementation as shown in as shown in FIGS. 3 and 4 respectively. The patches may be employed for targeted delivery of the microbial cocktail/antisense/compounds and other solutions proposed above. As shown in FIG. 3, the mosaic patch in the first implementation includes synthetic acid mantle, Lipoic acid or lipoic acid based ointment and a chelating agent. Further, as shown in FIG. 4 the mosaic patch in second implementation includes lipoic acid or lipoic acid based ointment and allosteric specific inhibitor/antisense-RNA placed on opposite corners of a square (where the squares are a component of a micro/nano grid payload base).

According to an embodiment of the disclosure, the method 100 can also be explained with the help of an example as follows.

Lipoic acid is an anti-inflammatory and hydrating topical medicine to treat skin-associated autoimmune inflammatory diseases. There are several biotic factors, which affects its bioavailability and penetration. Hence two separate publicly available datasets were analysed to validate and suggest appropriate solutions. The publicly available database was used for identifying the abundance in terms of KEGG Pathway Ids corresponding to the lipoic acid metabolic pathway. It was evident that there existed a differential status in the lipoic acid metabolic pathway, specifically in the microbial lipoic acid synthesis and salvage systems which validated that a biotic (microbial) factor affecting the bioavailability and penetration of lipoic acid.

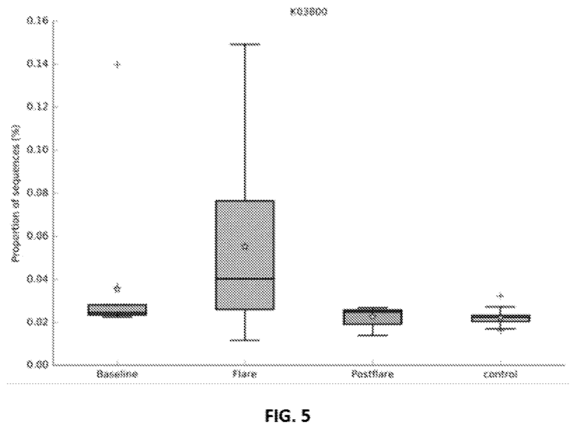

FIG. 5 to FIG. 11 shows the differential status and further solutions derived based on the following analysis.

FIG. 5 shows boxplot representation of the abundance of K03800 (involved in lipoic acid salvage, a component of lipoic acid metabolism in KEGG Pathways) in antecubital crease (Ac) samples obtained from control, and from Atopic Dermatitis (AD) affected subjects (at three stages of disease prominence i.e. baseline disease, flared up disease, post flare treated). The salvage system is apparently enhanced significantly in case of a disease flare up in AD. Lipoic acid bioavailability is reduced due to microbial salvage of available lipoic acid on skin (P-value 1.32 e-3).

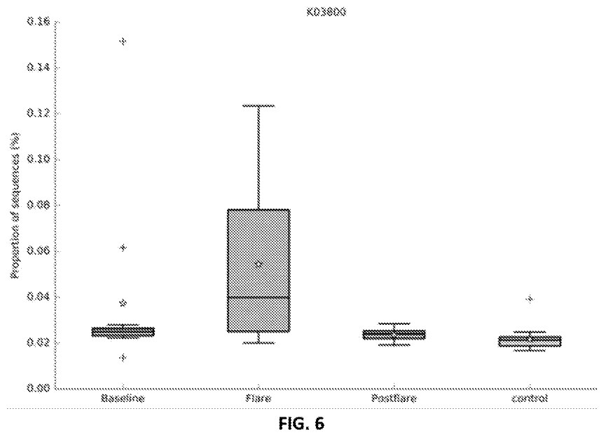

FIG. 6 shows boxplot representation of the abundance of K03800 (involved in lipoic acid salvage, a component of lipoic acid metabolism in KEGG Pathways) in popliteal creases (Pc) samples obtained from control, and from Atopic Dermatitis (AD) affected subjects (at three stages of disease prominence i.e baseline disease, flared up disease, post flare treated). The salvage system is apparently enhanced significantly in case of a disease flare up in AD. Lipoic acid bioavailability is reduced due to microbial salvage of available lipoic acid on skin (P-value 1.82 e-3).

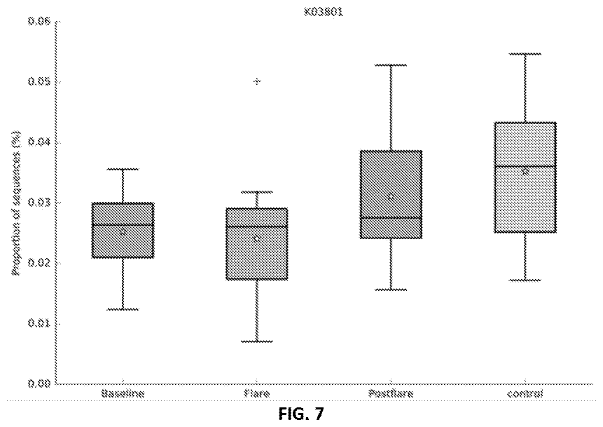

FIG. 7 shows a boxplot representation of the abundance of K03801 (involved in lipoic acid synthesis, a component of lipoic acid metabolism in KEGG Pathways) in Ac samples obtained from control, and from AD affected subjects (at three stages of disease prominence i.e. baseline disease, flared up disease, post flare treated). The Lipoic acid synthesis is apparently reduced significantly in case of a disease flare up in AD. Lipoic acid bioavailability is thus reduced due to low microbial synthesis of lipoic acid (P-value 0.014).

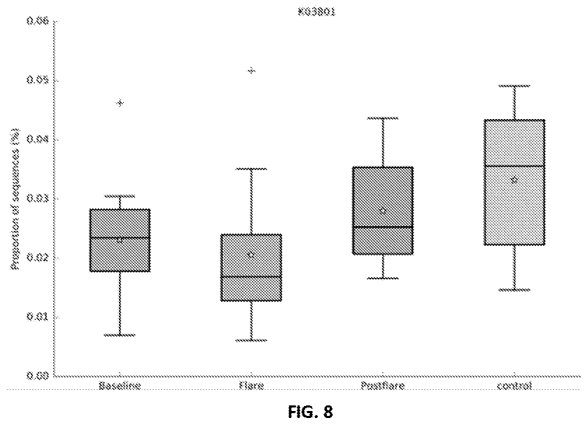

FIG. 8 shows a boxplot representation of the abundance of K03801 (involved in lipoic acid synthesis, a component of lipoic acid metabolism in KEGG Pathways) in Pc samples obtained from control, and from AD affected subjects (at three stages of disease prominence i.e. baseline disease, flared up disease, post flare treated). The Lipoic acid synthesis is apparently reduced significantly in case of a disease flare up in AD. Lipoic acid bioavailability is thus reduced due to low microbial synthesis of lipoic acid (P-value 9.45e-3).

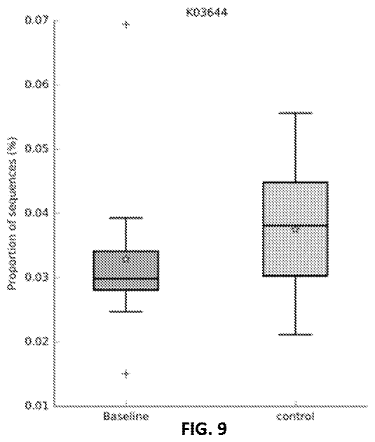

FIG. 9 shows a boxplot representation of the abundance of K03644 (involved in lipoic acid synthesis, a component of lipoic acid metabolism in KEGG Pathways) in Ac samples obtained from control, and from AD affected subjects (baseline). The Lipoic acid synthesis is apparently reduced in case of baseline disease in AD. Lipoic acid bioavailability is thus reduced due to low microbial synthesis of lipoic acid.

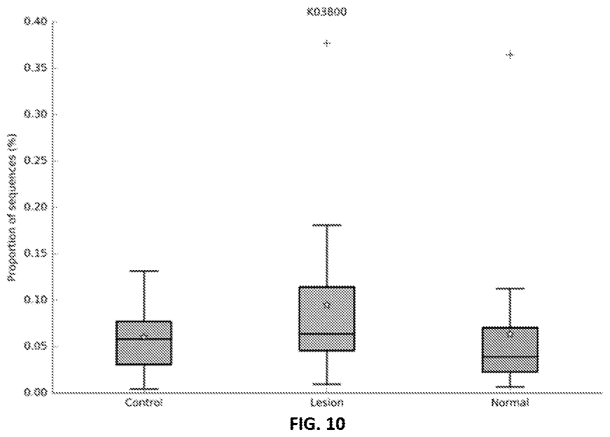

FIG. 10 shows a boxplot representation of the abundance of K03800 (involved in lipoic acid salvage, a component of lipoic acid metabolism in KEGG Pathways) in Psoriasis samples obtained from control, and from Psoriasis affected subjects (from affected site and unaffected ‘normal’ site). The salvage system is minutely enhanced in case of psoriasis affected sites. Lipoic acid bioavailability is reduced due to microbial salvage of available lipoic acid on skin.

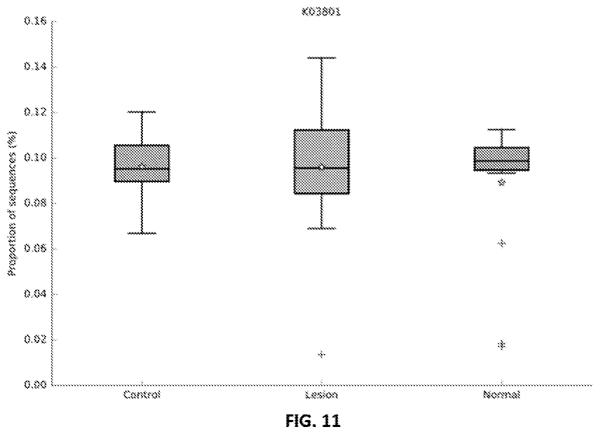

FIG. 11 shows a boxplot representation of the abundance of K03801 (involved in lipoic acid synthesis, a component of lipoic acid metabolism in KEGG Pathways) in Psoriasis samples obtained from control, and from Psoriasis affected subjects (from affected site and unaffected ‘normal’ site). The lipoic acid synthesis system is minutely reduced in case of psoriasis affected sites. Lipoic acid bioavailability is reduced due to low microbial synthesis of lipoic acid.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problems related to application of a lipoic acid based topical ointment efficacy in skin-penetration as well as its bioavailability after application on skin. The embodiment, thus provides a method and composition for microbiome based amelioration of skin associated autoimmune inflammatory diseases for a person.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

1. A method for preparing compositions for amelioration of skin-associated autoimmune inflammatory diseases for a person, the method comprising:
collecting sample from a lesional skin site of the person;
collecting sample from a healthy skin site of the person;
extracting microbial DNA from the collected healthy skin site and lesional skin site samples, wherein the extracted microbial DNA corresponds to genetic material extracted from a plurality of microbes or a subset of the plurality of microbes inhabiting on the skin of the person;
obtaining microbial sequence data by performing microbiome sequencing of:
the extracted microbial DNA corresponding to the plurality of microbes of the person, or
the extracted microbial DNA corresponding to the subset of the plurality of microbes harboring one or more of pathways which are involved in biosynthesis of lipoic acid and upregulation of lipoic acid salvage, wherein the subset of the plurality of microbes mentioned in a first list, a second list, a third list, a fourth list, a fifth list, and a sixth list;
generating, via the one or more hardware processors, microbiome taxonomic profiles from the obtained microbial sequencing data, wherein the microbiome taxonomic profiles indicate one of an absolute abundance and a relative abundance of each of the plurality of microbes inhabiting the lesional skin site or the healthy skin site samples respectively;
computing, via the one or more hardware processors, a first ratio of relative abundances of the subset of the plurality of microbes within the microbiome taxonomic profile from the lesional skin site harboring one or more pathways involved in lipoic acid biosynthesis to that of the subset of the plurality of microbes within the microbiome taxonomic profile from the lesional skin site harboring one or more pathways involved in upregulation of lipoic acid salvage;
computing, via the one or more hardware processors, a second ratio of relative abundances of the subset of the plurality of microbes within the microbiome taxonomic profile from the healthy skin site harboring one or more pathways involved in lipoic acid biosynthesis to relative abundances of subset of the plurality of microbes within the microbiome taxonomic profile from the lesional skin site harboring the same pathways in lipoic acid biosynthesis;
computing, via the one or more hardware processors, a third ratio of relative abundances of subset of the plurality of microbes within the microbiome taxonomic profile from healthy skin site harboring one or more pathways in upregulation of lipoic acid salvage to relative abundances of subset of the plurality of microbes within the microbiome taxonomic profile from lesion skin site harboring the same pathways in upregulation of lipoic acid salvage, wherein the subsets of the plurality of microbes for the first ratio, the second ratio and the third ratio are derived using a decision making system containing decisions defined by a unique combination of protein domains comprising one or more of LIAS_N, BPL_lplA_lipB, Lip_prot_lig_C and GCV (Glycine Cleavage Domain) protein domain to decide whether the microbe synthesizes, salvages or performs both salvage and synthesis functions;
administering a first composition to the person if the first ratio is less than one, wherein the first composition comprises one or more of:
one or more of microbes enlisted in the first list and the second list as probiotic,
one or more compounds inducing favorable physical and chemical factors,
anti-sense RNA sequences, or
antimicrobials,
and wherein the first composition is configured to perform one or more of:
promoting the growth of biosynthesis microbes through favorable physical factors and by administration of one or more probiotic non-pathogenic microbes harboring lipoic acid biosynthetic pathway enlisted in the first list and the second list, wherein the biosynthesis microbes refer to one or more microbes harboring one or more lipoic acid biosynthesis pathways,
reducing the abundance of salvage microbes through competing microbes and antibiotics that target one or more salvaging microbes involved in upregulation of lipoic acid salvage as enlisted in the third list, wherein the salvage microbes refer to one or more microbes harboring one or more pathways involved in upregulation of lipoic acid salvage, or
managing an amplified state through a set of salvage system inhibiting factors enlisted in a seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list and the fifth list and the sixth list are not removed in case they are non-pathogenic commensal microbes;
administering a second composition if the second ratio is more than one, wherein the second composition comprises:
one or more of microbes enlisted in the first list and the second list as probiotic, wherein the second composition is configured to perform one or more of:
promoting the growth of biosynthesis microbes through favourable physical factors and probiotic non-pathogenic microbes harboring lipoic acid biosynthetic pathway and lacking the salvage pathway as enlisted in the first list and the second list, or
promoting the abundance of microbes enlisted in the first list and the second list; and
administering a third composition if the third ratio is less than 1, wherein the third composition comprises:
competing microbes against microbes enlisted in the third list, the fourth list and the fifth list,
compounds inducing favorable physical and chemical factors, anti-sense RNA sequences, or
antimicrobials, wherein the third composition is configured to perform one or more of:
managing the amplified state of salvage microbes through competing microbes and antibiotics that target salvaging microbes enlisted in the third list, or
managing the amplified state of salvage microbes through salvage system inhibiting factors enlisted in the seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list, the fifth list and the sixth list are not removed in case they are non-pathogenic commensal microbes,
wherein the first composition, the second composition and the third composition are configured using the first ratio, second ratio and the third ratio respectively, and wherein the ratios are computed using the decision making system to identify microbes having one or both of lipoic acid synthesizing and salvaging functions, and wherein the administration of the first composition, the second composition and the third composition improves lipoic acid bioavailability for the person.

2. The method according to claim 1, wherein
the first list is a set of non-pathogenic microbes capable of thriving on skin and synthesizing lipoic acid,
the second list is a set of non-pathogenic lipoic acid synthesizing microbes other than that mentioned in first list,
the third list is a set of pathogenic salvage microbes which can be targeted by antibiotics,
the fourth list is a set of skin inhabiting microbes capable of degrading lipoic acid,
the fifth list is a set of skin inhabiting microbes, capable of degrading lipoic acid, other than those listed in the fourth list,
the sixth list is a set of non-pathogenic skin inhabiting microbes capable of synthesizing as well as degrading lipoic acid, and
the seventh list is one or more of synthetic acid mantle co-factors, anti-sense RNA sequences based inhibition factors and allosteric inhibition factors.

3. The method according to claim 1, wherein the microbes enlisted in the first list, the second list, the third list, the fourth list, the fifth list, and the sixth list are identified through following steps:
enlisting, via the one or more hardware processors, the plurality of skin inhabiting microbes and microbes capable of inhabiting skin are determined using one or more of a combination of laboratory based, -mining, machine learning techniques or in-silico methods;
profiling, via the one or more hardware processors, genomes of the enlisted plurality of microbes to identify a plurality of proteins involved in one or more of lipoic acid biosynthesis pathways or lipoic acid salvage pathways in the genomes;
identifying, via the one or more hardware processors, a plurality of protein domains corresponding to each of the identified plurality of proteins;
creating a domain matrix, via the one or more hardware processors, wherein column headers of the domain matrix correspond to individual enlisted genomes and row indices correspond to identified protein domains, and wherein a value of one is assigned to the enlisted genome if a domain out of the plurality of domains is present in the said genome else a value of zero is assigned;
deriving, via the one or more hardware processors, a pathway matrix from the domain matrix, via the one or more hardware processors, wherein for each of the given genomes represented in the domain matrix, a specific pathway is ascertained to be expressed in the genome if all domain(s) constituting the pathway indicate a value of 1 in the created domain matrix
identifying, via the one or more hardware processors, using the derived domain matrix and the pathway matrix, the microbes which are involved in different branch points of lipoic acid metabolism using a decision making system derived knowledgebase, wherein the decision making system derived knowledgebase contains different branch point definitions for different skin microbes, and wherein the mentioned branch point definitions refer to a
capability of a microbe to only synthesize lipoic acid
capability of a microbe to salvage lipoic acid
capability of a microbe to both synthesize and salvage lipoic acid,
wherein the branch point definitions are derived using a pattern of co-occurrence of the domains constituting the lipoic acid pathway as well as their presence in genomic neighborhood of each other, and wherein genomic neighborhood signifies the importance of co-occurrence of two domains in proximity to each other for the pathway to be functional.

4. (canceled)

5. The method according to claim 1, wherein the first composition, the second composition and the third composition is applied in the form of one or a plurality of skin-patches, wherein the plurality of skin-patches is employed for targeted delivery of the composition.

6. The method according to claim 5, wherein a first patch out of the plurality of patches is a mosaic patch comprises of a synthetic acid mantle, lipoic acid based ointment and a chelating agent.

7. The method according to claim 5, wherein a second patch out of the plurality of patches is the mosaic patch comprises lipoic acid based ointment and an allosteric specific inhibitor, antisense-RNA placed on opposite corners of a square of the second patch.

8. The method according to claim 1, wherein the skin associated auto-immune inflammatory diseases comprises one or more of atopic dermatitis, psoriasis or eczema.

9. The method according to claim 2, wherein the first list comprises one or more of: Corynebacterium efficiens, Corynebacterium glutamicum, Corynebacterium variabile, Corynebacterium callunae, Propionibacterium freudenreichii, Burkholderia vietnamiensis, Pseudomonas poae, Methylobacillus flagellatus, Erwinia tasmaniensis, Erwinia billingiae, Rhodoferax ferrireducens, Cupriavidus necator.

10. The method according to claim 2, wherein the second list comprises one or more of: Brachybacterium faecium, Corynebacterium efficiens, Corynebacterium glutamicum, Corynebacterium halotolerans, Salinispora tropica, Streptosporangium roseum, Corynebacterium variabile, Mycobacterium gilvum, Rubrobacter xylanophilus, Cellulomonas flavigena, Corynebacterium callunae, Sulfobacillus acidophilus, Microbacterium testaceum, Mycobacterium vanbaalenii, Mycobacterium indicuspranii, Propionibacterium freudenreichii, Nitrosococcus oceani, Nitrosococcus halophilus, Methylibium petroleiphilum, Methyloteneram obilis, Marinomonas mediterranea, Marinomonas posidonica, Cupriavidus necator, Shewanella amazonensis, Rhodoferax ferrireducens, Shewanella oneidensis, Shewanella woodyi, Shewanella violacea, Burkholderia vietnamiensis, Erwinia billingiae, Erwinia tasmaniensis, Thioalkalimicrobium cyclicum, Ramlibacter tataouinensis, Polaromonas naphthalenivorans, Alicycliphilus denitrificans, Aliivibrio fischeri, Buchnera aphidicola, Chromohalobacter salexigens, Psychromonas ingrahamii, Tolumonas auensis, Ferrimonas balearica, Halomonas elongate, Idiomarina loihiensis, Halothiobacillus neapolitanus, Methylobacillus flagellates, Thiomicrospira crunogena, Pseudomonas poae, Photobacterium profundum and Leptothrix cholodnii.

11. The method according to claim 2, wherein the third list comprises one or more of: Acholeplasma brassicae, Acholeplasma oculi, Acholeplasma palmae, Achromobacter denitrificans, Achromobacter xylosoxidans, Aerococcus urinae, Aerococcus urinaeequi, Aeromonas hydrophila, Aeromonas salmonicida, Aeromonas veronii, Bacillus anthracis, Bacillus cereus, Bacillus cytotoxicus, Bacillus infantis, Bacillus subtilis, Bacillus thuringiensis, Bordetella avium, Bordetella petrii, Brevibacillus brevis, Clostridioides difficile, Clostridium botulinum, Clostridium saccharolyticum, Clostridium tetani, Cronobacter condimenti, Cronobacter malonaticus, Cronobacter turicensis, Enterococcus casseliflavus, Enterococcus faecalis, Enterococcus faecium, Enterococcus hirae, Erysipelothrix larvae, Erysipelothrix rhusiopathiae Pathogenic, Escherichia fergusonii, Halobacteriovorax marinus, Klebsiella pneumoniae, Kluyvera intermedia, Lactobacillus fermentum, Lactobacillus parabuchneri, Leclercia adecarboxylata, Leptotrichia buccalis, Leuconostoc garlicum, Leuconostoc lactis, Leuconostoc mesenteroides, Listeria ivanovii, Listeria monocytogenes, Macrococcus caseolyticus, Melissococcus plutonius, Mesoplasma forum, Mycoplasma agalactiae, Mycoplasma bovis, Mycoplasma bovoculi, Mycoplasma capricolum, Mycoplasma cynos, Mycoplasma fermentans, Mycoplasma gallisepticum, Mycoplasma genitalium, Mycoplasma hyopneumoniae, Mycoplasma leachii, Mycoplasma mobile, Mycoplasma mycoides, Mycoplasma penetrans, Mycoplasma pneumoniae, Mycoplasma putrefaciens, Mycoplasma synoviae, Paenibacillus larvae, Pantoea agglomerans, Pantoea ananatis, Pectobacterium carotovorum, Prevotella enoeca, Prevotella intermedia, Rahnella aquatilis, Raoultella ornithinolytica, Salmonella enterica, Shigella boydii, Shigella dysenteriae, Shigella flexneri, Shigella sonnei, Spiroplasma apis, Spiroplasma citri, Spiroplasma culicicola, Spiroplasma mirum, Staphylococcus aureus, Staphylococcus haemolyticus, Staphylococcus lugdunensis, Staphylococcus pasteuri, Staphylococcus pseudintermedius, Staphylococcus saprophyticus, Streptobacillus moniliformis, Streptococcus acidominimus, Streptococcus anginosus, Streptococcus constellatus, Streptococcus dysgalactiae, Streptococcus equi, Streptococcus gallolyticus, Streptococcus gordonii, Streptococcus infantarius, Streptococcus iniae, Streptococcus lutetiensis, Streptococcus mitis, Streptococcus mutans, Streptococcus oralis, Streptococcus parasanguinis, Streptococcus parauberis, Streptococcus pasteurianus, Streptococcus pneumoniae, Streptococcus pyogenes, Streptococcus sanguinis, Streptococcus suis, Streptococcus uberis, Thermoanaerobacterium thermosaccharolyticum, Treponema denticola, Treponema pedis, Yersinia enterocolitica, Yersinia pestis, Yersinia pseudotuberculosis.

12. The method according to claim 2, wherein the fourth list comprises one or more of: Auricoccus indicus, Staphylococcus epidermidis, Pediococcus acidilactici, Clostridium kluyveri, Lactobacillus brevis, Lactobacillus buchneri, Lactobacillus koreensis, Lactococcus lactis, Streptococcus agalactiae.

13. The method according to claim 2, wherein the fifth list comprises one or more of: Acetohalobium arabaticum, Acholeplasma laidlawii, Aerococcus urinaehominis, Auricoccus indicus, Bacillus amyloliquefaciens, Bacillus atrophaeus, Bacillus cellulosilyticus, Bacillus clausii, Bacillus coagulans, Bacillus halodurans, Bacillus licheniformis, Bacillus megaterium, Bacillus mycoides, Bacillus pumilus, Bacillus toyonensis, Bacillus velezensis, Bdellovibrio bacteriovorus, Carnobacterium maltaromaticum, Carnobacterium sp., Clostridium propionicum, Deinococcus proteolyticus, Desulfarculus baarsii, Desulfitobacterium hafniense, Desulfitobacterium metallireducens, Desulfomicrobium baculatum, Desulfotomaculum ruminis, Desulfovibrio magneticus, Enterococcus mundtii, Escherichia coli, Eubacterium limosum, Exiguobacterium antarcticum, Exiguobacterium sibiricum, Glutamicibacter arilaitensis, Halothermothrix orenii, Hydrogenobacter thermophiles, Ilyobacter polytropus, Intrasporangium calvum, Kosakonia oryzae, Lactobacillus acidipiscis, Lactobacillus casei, Lactobacillus paracasei, Lactobacillus paraplantarum, Lactobacillus plantarum, Lactobacillus reuteri, Lactobacillus rhamnosus, Lactobacillus sakei, Lactobacillus salivarius, Leadbetterella byssophila, Leuconostoc carnosum, Leuconostoc citreum, Leuconostoc gelidum, Leuconostoc kimchi, Listeria innocua, Listeria seeligeri, Listeria welshimeri, Lysinibacillus sphaericus, Meiothermus silvanus, Oenococcus oeni. Oscillibacter valericigenes, Owenweeksia hongkongensis, Paenibacillus bovis, Paenibacillus mucilaginosus, Paenibacillu sterrae, Pantoe avagans, Pediococcus pentosaceus, Planococcus donghaensis, Shimwellia blattae, Sporosarcina psychrophila, Staphylococcus carnosus, Staphylococcus epidermidis, Staphylococcus piscifermentans, Staphylococcus warneri, Streptococcus salivarius, Streptococcus thermophiles, Symbiobacterium thermophilum, Syntrophomonas wolfei, Tetragenococcushalophilus, Weissellakoreensis, Yersinia intermedia, Zunongwangia profunda.

14. The method according to claim 2, wherein the sixth list comprises one or more of: Clostridium kluyveri, Clostridium ljungdahlii, Oceanobacillus iheyensis, Actinobacillus succinogenes and Enterobacter lignolyticus.

15. (canceled)

16. A composition for amelioration of skin-associated autoimmune inflammatory diseases, the composition comprising one or more of a first composition, a second composition or a third composition, wherein
the first composition comprising one or more of:
one or more of microbes enlisted in the first list and the second list as probiotic,
one or more compounds inducing favorable physical and chemical factors,
anti-sense RNA sequences, or
antimicrobials, wherein the first composition is administered if a first ratio is less than one,
the second composition comprising one or more of microbes enlisted in the first list and the second list as probiotic, wherein the second composition is administered if a second ratio is more than one, and
the third composition comprising one or more of:
competing microbes against microbes enlisted in the third list, the fourth list and the fifth list,
compounds inducing favorable physical and chemical factors,
anti-sense RNA sequences, or
antimicrobials, wherein the third composition is administered if a third ratio is less than one, —
and wherein the first ratio, the second ration, and the third ratio is computed as follows:
collecting sample from a lesional skin site of the person;
collecting sample from a healthy skin site of the person;
extracting microbial DNA from the collected healthy skin site and lesional skin site samples, wherein the extracted microbial DNA corresponds to genetic material extracted from a plurality of microbes or a subset of the plurality of microbes inhabiting on the skin of the person;
obtaining microbial sequence data by performing microbiome sequencing of:
the extracted microbial DNA corresponding to the plurality of microbes of the person, or
the extracted microbial DNA corresponding to the subset of the plurality of microbes harboring one or more of pathways which are involved in biosynthesis of lipoic acid and upregulation of lipoic acid salvage, wherein the subset of the plurality of microbes mentioned in a first list, a second list, a third list, a fourth list, a fifth list, and a sixth list,
wherein the first composition, the second composition and the third composition are configured using the first ratio, second ratio and the third ratio respectively, and wherein the ratios are computed using the decision making system to identify microbes having one or both of lipoic acid synthesizing and salvaging functions, and wherein the administration of the first composition, the second composition and the third composition improves lipoic acid bioavailability for the person;
generating microbiome taxonomic profiles from the obtained microbial sequencing data, wherein the microbiome taxonomic profiles indicate one of an absolute abundance and a relative abundance of each of the plurality of microbes inhabiting the lesional skin site or the healthy skin site samples respectively;
computing the first ratio of relative abundances of the subset of the plurality of microbes within the microbiome taxonomic profile from the lesional skin site harboring one or more pathways involved in lipoic acid biosynthesis to that of the subset of the plurality of microbes within the microbiome taxonomic profile from the lesional skin site harboring one or more pathways involved in upregulation of lipoic acid salvage;
computing the second ratio of relative abundances of the subset of the plurality of microbes within the microbiome taxonomic profile from the healthy skin site harboring one or more pathways involved in lipoic acid biosynthesis to relative abundances of subset of the plurality of microbes within the microbiome taxonomic profile from the lesional skin site harboring the same pathways in lipoic acid biosynthesis;
computing the third ratio of relative abundances of subset of the plurality of microbes within the microbiome taxonomic profile from healthy skin site harboring one or more pathways in upregulation of lipoic acid salvage to relative abundances of subset of the plurality of microbes within the microbiome taxonomic profile from lesion skin site harboring the same pathways in upregulation of lipoic acid salvage, wherein the subsets of the plurality of microbes for the first ratio, the second ratio and the third ratio are derived using a decision making system containing decisions defined by a unique combination of protein domains comprising one or more of LIAS_N, BPL_IplA_lipB, Lip_prot_lig_C and GCV (Glycine Cleavage Domain) protein domain to decide whether the microbe synthesizes, salvages or performs both salvage and synthesis functions.

17. The composition according to claim 16, wherein
the first composition is configured to perform one or more of:
promoting the growth of biosynthesis microbes through favorable physical factors and by administration of one or more probiotic non-pathogenic microbes harboring lipoic acid biosynthetic pathway enlisted in the first list and the second list, wherein the biosynthesis microbes refer to one or more microbes harboring one or more lipoic acid biosynthesis pathway,
reducing the abundance of salvage microbes through competing microbes and antibiotics that target one or more salvaging microbes involved in upregulation of lipoic acid salvage as enlisted in the third list, wherein the salvage microbes refer to one or more microbes harboring one or more pathways involved in upregulation of lipoic acid salvage, or
managing an amplified state through a set of salvage system inhibiting factors enlisted in a seventh list, wherein the seventh list comprises one of synthetic acid mantle co-factors, an anti-sense RNA sequences based inhibition factors and allosteric inhibition factors, wherein the inhibiting factors ensure that microbes enlisted in the fourth list and the fifth list and the sixth list are not removed in case they are non-pathogenic commensal microbes,
the second composition is configured to perform one or more of:
promoting the growth of biosynthesis microbes through favourable physical factors and probiotic non-pathogenic microbes harboring lipoic acid biosynthetic pathway and lacking the salvage pathway as enlisted in the first list and the second list, or
promoting the abundance of microbes enlisted in the first list and the second list, and
the third composition is configured to perform one or more of:
managing the amplified state of salvage microbes through competing microbes and antibiotics that target salvaging microbes enlisted in the third list, or
managing the amplified state of salvage microbes through salvage system inhibiting factors enlisted in the seventh list, wherein the seventh list comprises one of synthetic acid mantle co-factors, anti-sense RNA sequences based inhibition factors and allosteric inhibition factors, wherein the inhibiting factors ensure that microbes enlisted in the fourth list, the fifth list and the sixth list are not removed in case they are non-pathogenic commensal microbes.

18. The composition according to claim 16, wherein
the first list is a set of non-pathogenic microbes capable of thriving on skin and synthesizing lipoic acid,
the second list is a set of non-pathogenic lipoic acid synthesizing microbes other than that mentioned in first list,
the third list is a set of pathogenic salvage microbes which can be targeted by antibiotics,
the fourth list is a set of skin inhabiting microbes capable of degrading lipoic acid,
the fifth list is a set of skin inhabiting microbes, capable of degrading lipoic acid, other than those listed in the fourth list,
the sixth list is a set of non-pathogenic skin inhabiting microbes capable of synthesizing as well as degrading lipoic acid, and
the seventh list is one or more of synthetic acid mantle co-factors, anti-sense RNA sequences based inhibition factors and allosteric inhibition factors.

19. The composition according to claim 16, wherein the first list comprises one or more of: Corynebacterium efficiens, Corynebacterium glutamicum, Corynebacterium variabile, Corynebacterium callunae, Propionibacterium freudenreichii, Burkholderia vietnamiensis, Pseudomonas poae, Methylobacillus flagellatus, Erwinia tasmaniensis, Erwinia billingiae, Rhodoferax ferrireducens, Cupriavidus necator.

20. The composition according to claim 16, wherein the second list comprises one or more of: Brachybacterium faecium, Corynebacterium efficiens, Corynebacterium glutamicum, Corynebacterium halotolerans, Salinispora tropica, Streptosporangium roseum, Corynebacterium variabile, Mycobacterium gilvum, Rubrobacter xylanophilus, Cellulomonas flavigena, Corynebacterium callunae, Sulfobacillus acidophilus, Microbacterium testaceum, Mycobacterium vanbaalenii, Mycobacterium indicuspranii, Propionibacterium freudenreichii, Nitrosococcus oceani, Nitrosococcus halophilus, Methylibium petroleiphilum, Methyloteneram obilis, Marinomonas mediterranea, Marinomonas posidonica, Cupriavidus necator, Shewanella amazonensis, Rhodoferax ferrireducens, Shewanella oneidensis, Shewanella woodyi, Shewanella violacea, Burkholderia vietnamiensis, Erwinia billingiae, Erwinia tasmaniensis, Thioalkalimicrobium cyclicum, Ramlibacter tataouinensis, Polaromonas naphthalenivorans, Alicycliphilus denitrificans, Aliivibrio fischeri, Buchnera aphidicola, Chromohalobacter salexigens, Psychromonas ingrahamii, Tolumonas auensis, Ferrimonas balearica, Halomonas elongate, Idiomarina loihiensis, Halothiobacillus neapolitanus, Methylobacillus flagellates, Thiomicrospira crunogena, Pseudomonas poae, Photobacterium profundum and Leptothrix cholodnii.

21. The composition according to claim 16, wherein the third list comprises one or more of: Acholeplasma brassicae, Acholeplasma oculi, Acholeplasma palmae, Achromobacter denitrificans, Achromobacter xylosoxidans, Aerococcus urinae, Aerococcus urinaeequi, Aeromonas hydrophila, Aeromonas salmonicida, Aeromonas veronii, Bacillus anthracis, Bacillus cereus, Bacillus cytotoxicus, Bacillus infantis, Bacillus subtilis, Bacillus thuringiensis, Bordetella avium, Bordetella petrii, Brevibacillus brevis, Clostridioides difficile, Clostridium botulinum, Clostridium saccharolyticum, Clostridium tetani, Cronobacter condimenti, Cronobacter malonaticus, Cronobacter turicensis, Enterococcus casseliflavus, Enterococcus faecalis, Enterococcus faecium, Enterococcus hirae, Erysipelothrix larvae, Erysipelothrix rhusiopathiae Pathogenic, Escherichia fergusonii, Halobacteriovorax marinus, Klebsiella pneumoniae, Kluyvera intermedia, Lactobacillus fermentum, Lactobacillus parabuchneri, Leclercia adecarboxylata, Leptotrichia buccalis, Leuconostoc garlicum, Leuconostoc lactis, Leuconostoc mesenteroides, Listeria ivanovii, Listeria monocytogenes, Macrococcus caseolyticus, Melissococcus plutonius, Mesoplasma forum, Mycoplasma agalactiae, Mycoplasma bovis, Mycoplasma bovoculi, Mycoplasma capricolum, Mycoplasma cynos, Mycoplasma fermentans, Mycoplasma gallisepticum, Mycoplasma genitalium, Mycoplasma hyopneumoniae, Mycoplasma leachii, Mycoplasma mobile, Mycoplasma mycoides, Mycoplasma penetrans, Mycoplasma pneumoniae, Mycoplasma putrefaciens, Mycoplasma synoviae, Paenibacillus larvae, Pantoea agglomerans, Pantoea ananatis, Pectobacterium carotovorum, Prevotella enoeca, Prevotella intermedia, Rahnella aquatilis, Raoultella ornithinolytica, Salmonella enterica, Shigella boydii, Shigella dysenteriae, Shigella flexneri, Shigella sonnei, Spiroplasma apis, Spiroplasma citri, Spiroplasma culicicola, Spiroplasma mirum, Staphylococcus aureus, Staphylococcus haemolyticus, Staphylococcus lugdunensis, Staphylococcus pasteuri, Staphylococcus pseudintermedius, Staphylococcus saprophyticus, Streptobacillus moniliformis, Streptococcus acidominimus, Streptococcus anginosus, Streptococcus constellatus, Streptococcus dysgalactiae, Streptococcus equi, Streptococcus gallolyticus, Streptococcus gordonii, Streptococcus infantarius, Streptococcus iniae, Streptococcus lutetiensis, Streptococcus mitis, Streptococcus mutans, Streptococcus oralis, Streptococcus parasanguinis, Streptococcus parauberis, Streptococcus pasteurianus, Streptococcus pneumoniae, Streptococcus pyogenes, Streptococcus sanguinis, Streptococcus suis, Streptococcus uberis, Thermoanaerobacterium thermosaccharolyticum, Treponema denticola, Treponema pedis, Yersinia enterocolitica, Yersinia pestis, Yersinia pseudotuberculosis.

22. The composition according to claim 16, wherein the fourth list comprises one or more of: Auricoccus indicus, Staphylococcus epidermidis, Pediococcus acidilactici, Clostridium kluyveri, Lactobacillus brevis, Lactobacillus buchneri, Lactobacillus koreensis, Lactococcus lactis, Streptococcus agalactiae.

23-25. (canceled)
